(19) United States
(12) Patent Application Publication
(10) Pub. No.: US 2015/0290439 A1
(43) Pub. Date: Oct. 15, 2015

(54) METHODS FOR BILATERAL CENTRAL AUTONOMIC NEUROMODULATION

(71) Applicants: Stephen Eldredge, South Jordan, UT (US); Richard John Harp, Spartanburg, SC (US); Wade Cooper, Ann Arbor, MI (US)

(72) Inventors: Stephen Eldredge, South Jordan, UT (US); Richard John Harp, Spartanburg, SC (US); Wade Cooper, Ann Arbor, MI (US)

(21) Appl. No.: 14/683,542

(22) Filed: Apr. 10, 2015

Related U.S. Application Data
(60) Provisional application No. 61/979,420, filed on Apr. 14, 2014.

(51) Int. Cl. A61M 31/00; A61M 19/00

(52) U.S. Cl. CPC A61M 31/00; A61M 19/00; A61M 2205/32; A61M 2210/0618; A61M 2205/6018

(57) ABSTRACT

The invention provides a system and method for pain treatment in which a medication is delivered with good localization to the SPG and to the maxillary division of the trigeminal nerve—known a V2. A treating physician uses a drug delivery device to insert a delivery catheter into nostril of a patient lying supine and orient the catheter with respect to the SPG and V2. The physician operates a mechanism on the catheter to express the medication and allows for gravity-influenced transport to bring the medication to the SPG and V2.

### CROSS-REFERENCE TO RELATED APPLICATION

This application claims the benefit of, and priority to, U.S. Provisional Application Ser. No. 61/979,420, filed Apr. 14, 2014, the contents of which are incorporated by reference.

### FIELD OF THE INVENTION

The invention relates to methods for treating pain such as from migraine headaches.

### BACKGROUND

A migraine headache can severely limit a person's ability to participate in and experience work, driving, enjoying lunch, or even spending time with the family. Currently there are few promising treatments available to a migraine patient. Nearly two thirds of patients discontinue prescription medications due to inadequate relief and unwanted side-effects.

One target for pain management has been the sphenopalatine ganglion (SPG), a cluster of nerve cells associated with the trigeminal nerve in the skull. The SPG is the largest collection of neurons in the head outside of the brain. Early attempts to treat pain by targeting the SPG included injecting cocaine through the patient's face with a long needle. Contemporary pain treatments involve applying lidocaine to the SPG via a long cotton swab inserted through the nose. Existing approaches are lacking because they do not ensure that the SPG is fully served by the medication. Pushing the cotton swab up the nose is something of a hunt-around-and-guess operation in which it is hoped that at least some of the medication gets to the SPG and is not just wiped onto other tissue.

### SUMMARY

The invention provides a system and method for pain treatment in which a medication is delivered with good localization to the SPG and to the maxillary division of the trigeminal nerve—known a V2. A treating physician uses a drug delivery device to insert a delivery catheter into nostril of a patient lying supine and orient the catheter with respect to the SPG and V2. The physician operates a mechanism on the catheter to express the medication and allows for gravity-influenced transport to bring the medication to the SPG and V2. By performing the operation in both nostrils, the physician can cause the medication to migrate into the bilateral pterygopalatine fossi and thus effect a bilateral medication of both SPG and both V2. The medication can include a suitable pain medication such as an anesthetic, a calcitonin gene related peptide (CGRP) antagonist, or some other neuromodulator. Because the pain medication provides a bilateral treatment effectively targeted to the SPG and the V2, the medication hits its target and provide effective relief from pain such as migraine headache pain. Methods of the invention can provide neuromodulation effects. Due to the effective pain relief provided by methods of the invention, a patient can enjoy a greatly improved quality of life by being fully engaged in life activities without interference from migraine pain.

In certain aspects, the invention provides a method of treating a patient for pain. The method includes inserting at least one catheter of a drug delivery device into a nostril of a patient lying supine, operating a delivery mechanism on a proximal portion of the drug delivery device to cause a medication to be pushed in a distal direction through the catheter, and delivering the medication out of an exit port on a distal end of the catheter and into a target site in the patient. This further includes medicating a maxillary branch of a trigeminal nerve (V2) and a sphenopalatine ganglion (SPG) by causing the medication to make contact with the V2 and the SPG. Preferably, in the drug delivery device, the catheter defines an extended body with the distal end in which the distal end includes a curved portion and the exit port. Preferably, the drug delivery device includes a handle at the proximal portion of the catheter, at least a first lumen in the catheter such that movement of an inner member delivers the medication through the first lumen and out of the exit port at the distal portion of the catheter, and a reservoir within the device for holding the medication. The proximal end of the drug delivery may have a marking on the handle that indicates an orientation of the curved portion of the catheter member.

The method may further include determining the orientation of the curved portion of the catheter member (e.g., by touching the marking on the handle) and optimizing the orientation (e.g., by manipulating the handle). It may be preferably to instruct the patient to remain supine for at least a minute to allow the medication to migrate into a pterygopalatine fossa. The inserting and operating steps may be performed for each of two nostrils of the patient. Thus the method includes causing the medication to make bilateral contact with both V2 and both SPG of the patient. The delivered medication may include a neuromodulator, an anesthetic, a calcitonin gene-related peptide (CGRP) receptor antagonist, an analgesic, others, or a combination thereof.

Optional features of the drug delivery device include a radiopaque marking band (e.g., located at the distal portion of the catheter), a gripping surface with textured portions for grasping the drug delivery device, a plunger member operable to apply pressure to the medication in the drug delivery device, or any combination thereof. In some embodiments, the medication provides a means for delivery of agents effective for repolarization of cranial sensory and parasympathetic pathways via trigeminal primary afferent neurons. In a preferred embodiment, the marking on the handle comprises a three-dimensional indicia configured to be detected via touch by a hand of an operator to indicate the orientation of the curved distal end of the catheter. The three-dimensional indicia may be shaped to represent the curved distal end of the catheter. The method optionally includes viewing instructions describing the method on information materials provided with the drug delivery device (e.g., the drug delivery device may be packed with instructions or a reference to instructions).

### BRIEF DESCRIPTION OF THE DRAWINGS

FIG. 1 shows a drug delivery device emplaced within a patient's nasal anatomy.

FIG. 2 shows cranial nerves addressed by methods of the invention.

FIG. 3 is a top side perspective view of a drug delivery device of one embodiment.

FIG. 4 is a top view of the drug delivery device.

FIG. 5A is a perspective view of a second embodiment of a drug delivery device.

FIG. 5B shows the device in an extended position.

FIG. 6A is a top view of a device of the second embodiment, extended position.

FIG. 6B is a side cutaway view of a device of the second embodiment.

FIG. 7A is shows the second embodiment of device with hub in an inserted position.

FIG. 7B is a side cutaway view of a drug delivery device of the second embodiment.

FIG. 8 is a side view of a drug delivery device of the first embodiment.

FIG. 9 is an opposite-side view of a drug delivery device.

FIG. 10 shows movement direction of the first embodiment of the drug delivery device.

FIG. 11 shows chemical that may be used.

FIG. 12 shows steps for intranasal treatment of a patient.

FIG. 13 diagrams methods of the invention.

### DETAILED DESCRIPTION

The present inventors have discovered that effecting neuromodulation can be accomplished by dispersion of medication over the middle turbinate—part of the bone shelf protruding into the breathing passage of the nose. The invention accordingly provides methods for pain treatment in which a medication is delivered over the middle turbinate and to the SPG and V2.

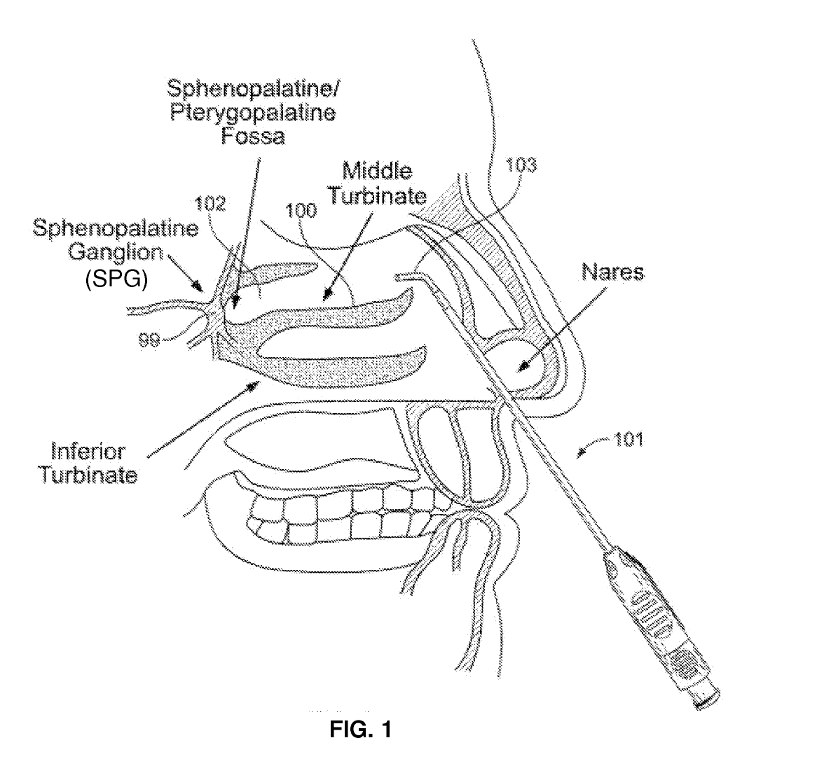

FIG. 1 shows use of a drug delivery device 101 to insert a delivery catheter into nostril of a patient lying supine and deliver medication from an exit port of the catheter over a middle turbinate 100. Visible in FIG. 1, the SPG 99 functions as a biologic “circuit” along with other neuronal cell bodies, including but not limited to those autonomic ganglia known as the Stellate Lumbar Sympathetic, Celiac Plexus (and others). By directly contacting and medicating SPG 99, a collective chemical repolarization of these autonomic ganglia is achieved. Physiologically, this type of collective chemical repolarization is analogous to the type of reset mechanism used to counter atrial fibrillation, when the heart undergoes electrical repolarization in cardioversion.

Methods of the invention include orienting the catheter 101 with respect to the SPG and V2 to disburse medication over the middle turbinate 100. The inventors have discovered that the injectate delivered through the drug delivery device 101 migrates under gravitational assistance into the bilateral pterygopalatine fossi 102. This remarkable discovery provides a mechanism for delivery of local anesthetics along with other drugs to make contact with and provide drug target interface with both the SPG and V2 of the trigeminal ganglion. This lateral migration of the injectate occurs into both sides of the face when the device 101 administered medication through both nostrils. Thus, the procedure may be referred to as a “bilateral central neuro-modulation, with bilateral SPG and V2 blockade.

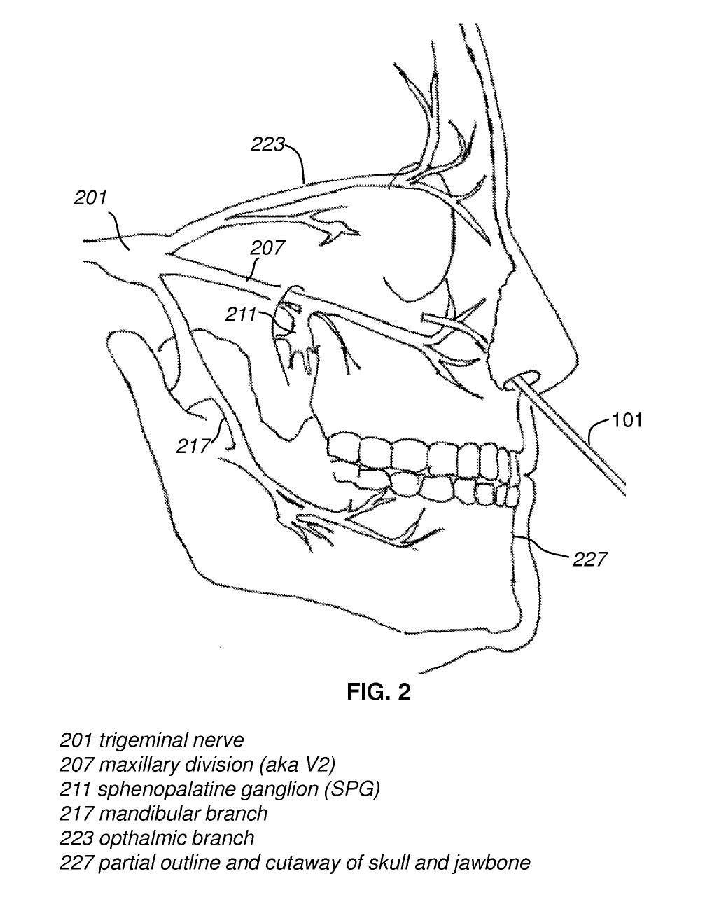

FIG. 2 illustrates to the maxillary division (V2) of the trigeminal nerve 201 and the SPG 211 among other cranial nerves, and shows how those nerves are oriented in the skull. Each cranial nerve is bilateral—paired and is present on both sides. The depicted cranial nerves are part of the peripheral nervous system which includes twelve pairs of cranial nerves numbered I-XII. The trigeminal nerves 201 is numbered V and is named for three heads (L: tri+ geminus).

The cranial nerves give rise to a number of ganglia—collections of the cell bodies of neurons in the nerves that are outside of the brain. These ganglia are both parasympathetic and sensory ganglia. After emerging from the brainstem, the cranial nerves travel within the skull, but must leave this bony compartment in order to reach their destinations. The maxillary division of the trigeminal nerve (V2) passes through a round foramen. It is understood that pain may follow the distribution of the maxillary or mandibular nerve (branches V2 and V3). Important anatomic components of migraine headaches include the meningeal vasculature, trigeminal nerves, and other notable areas in the brain stem. When these areas are sensitized beyond their threshold, a migraine is triggered. The maxillary division of the trigeminal nerve (V2) has sensory nociceptive fibers that travel through the SPG to innervate the roof of the mouth via the palatine nerves as well as the turbinates. The SPG plays an important role in the relationship between the sinus regions and migraine. The SPG is attached to the maxillary nerve of the trigeminal system and includes nociceptive fibers that travel through the SPG from V2. It is thought that migraines involve the activation of nociceptive pathways of the nose and sinus and the trigeminal autonomic system. Similarly, sinus lesions or infections can activate the trigeminal vascular complex implicating trigeminal activation and meningeal effects that lead to migraines and related symptoms. For at least those reasons, the SPG and V2 present attractive targets for pain treatment. Using methods of the invention, a physician can deliver medication bilaterally to the SPG and V2. The physician operates a mechanism on the catheter 101 to express the medication and allows for gravity-influenced transport to bring the medication to the SPG and V2.

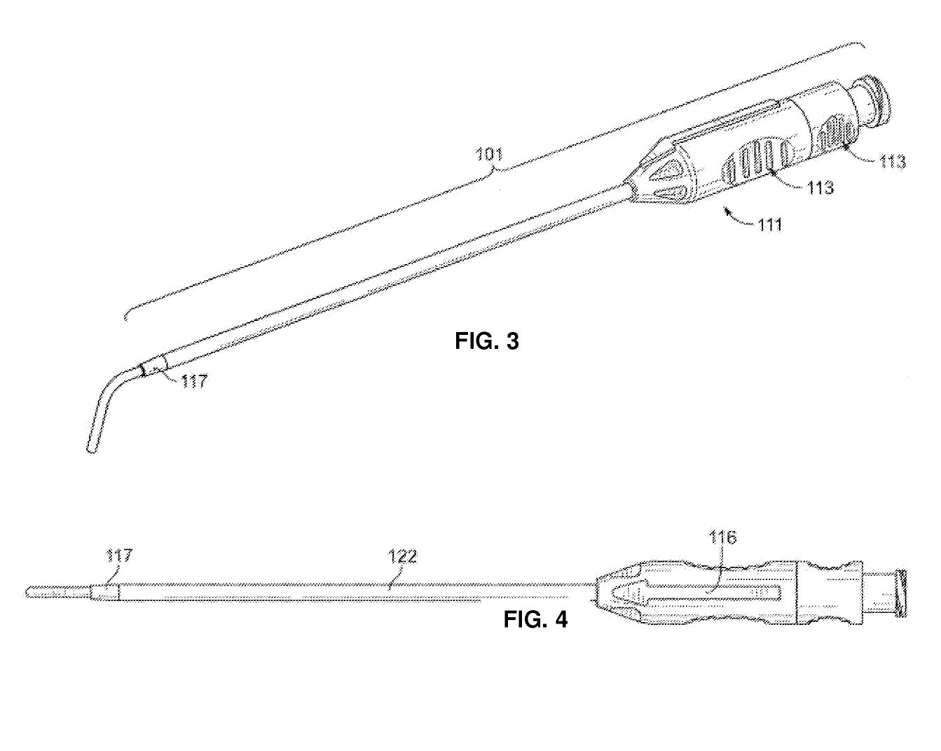

FIG. 3 shows a drug delivery device 101 suitable for use with the invention. Drug delivery device 101 may be provided by, for example, the device sold under the trademark SPHENOCATH by Dolor Technologies (Salt Lake City, Utah). Device 101 includes an ergonomic hub/handle 111, feature finger gripping means 113 placed on the side of device 101.

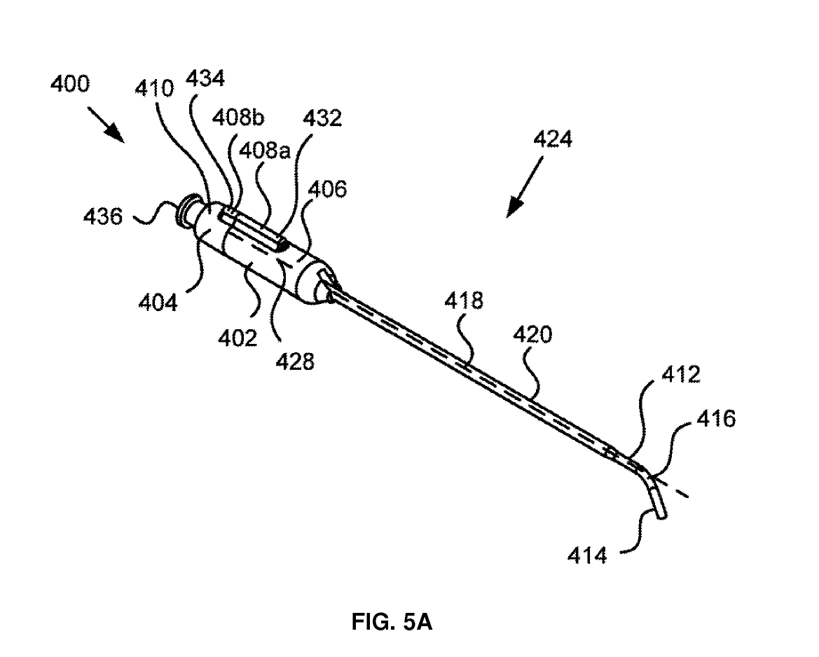

FIG. 4 shows directional arrow 116 and radiopaque marking tip 117, which is often made of tungsten (W). Those skilled in the art understand tungsten, tantalum and/or platinum may further be disposed within shaft 122 of device 101 for visualization under imaging technologies.

FIG. 4 is a ventral view, showing device 101 with its ergonomic hub/handle 111, radiopaque marker 117, and grooved arresting element 119. Infusion port 121 and medication delivery port 123 demonstrate a relationship between deflecting tip angle for delivery port 123.

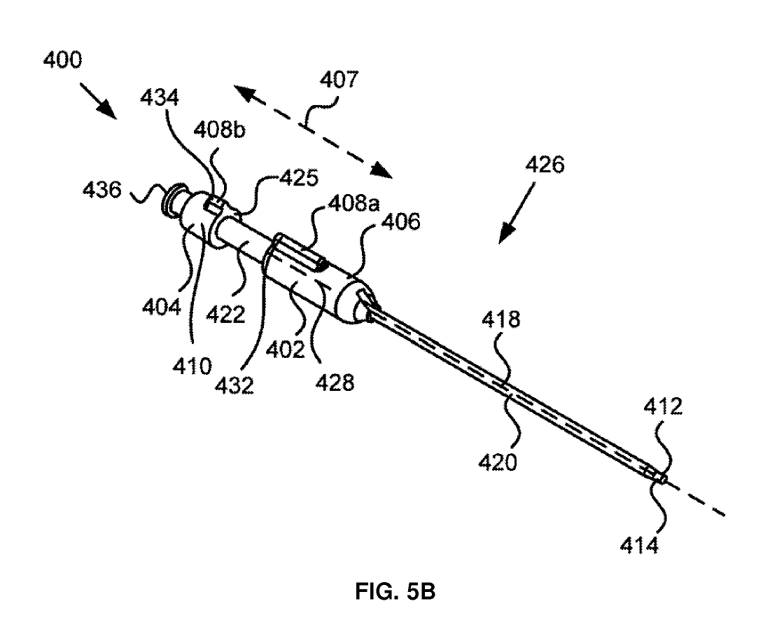

FIG. 5A is a perspective view of a second embodiment of a drug delivery device 424. FIG. 5A is a perspective view illustrating one embodiment of an apparatus 424 for facilitating intranasal treatment of a patient's sphenopalatine/pterygopalatine recess with a catheter hub 402 positioned in an inserted position within a sheath hub (ready for medication delivery) in accordance with the present subject matter. The drug delivery device 426 suits a method for treating a pain condition. The device includes a catheter member 418 defining an extended body 420 with a proximal portion and a distal portion, wherein the distal portion defines a delivery port 414, a handle 402 at the proximal portion of the catheter 426, and at least a first lumen 420 within the catheter. Movement of an inner member 422 of a delivery mechanism 400 results in delivery of a medication out of the delivery port 414 at the distal portion of the catheter. The device includes a reservoir 406 holding a medication.

The device may include an arresting element 434 on the handle, the arresting element comprising a groove and detent mechanism 408. In some embodiments, a portion of the resting element is pressed inward from the detent mechanism and slid in a proximal direction, causing the inner member 422 (such as a plunger-type member 436 on butt end 410 or a pump type member) to increase pressure on the medication in the reservoir 406, thereby sending the medication through a lumen 420 of the catheter such that the medication is delivered through the delivery port 414.

FIG. 5A depicts a perspective view of a second embodiment of an apparatus 400 for methods of the invention. The apparatus 400 includes the sheath hub 402, the catheter hub 404, a catheter 412, and a sheath 420. The apparatus 400 of FIG. 5A is depicted with a catheter hub 404 positioned in an inserted position 424 within a sheath hub 402.

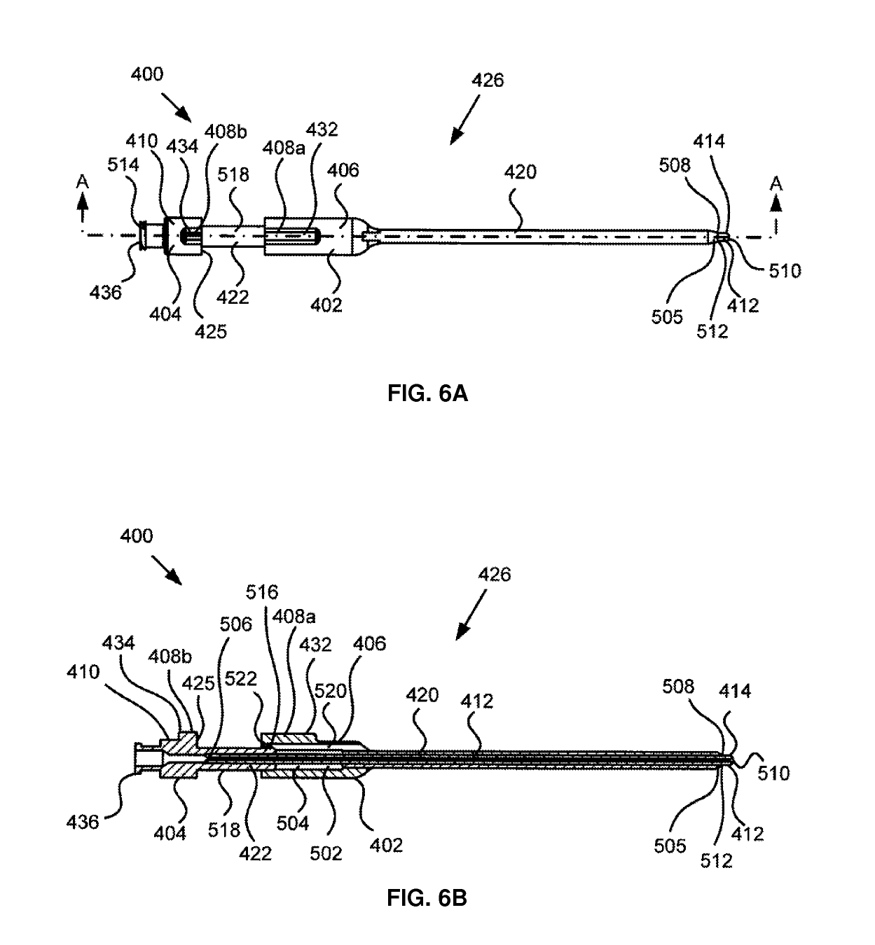

FIG. 5B depicts a perspective view of one embodiment of the apparatus 400 of FIG. 5A with the catheter hub 404 positioned in an extended position 426 within the sheath hub 402. The sheath hub 402 includes an exterior surface 406 that opposes an interior surface 502 (FIG. 6B). The interior surface 502 of the catheter hub 404 defines a catheter hub receiving space 504 (FIG. 6B). At least a portion 422 of the catheter hub 404 is received within the catheter hub receiving space 504 and the catheter hub 404 is repositionable along a longitudinal axis 428 of the sheath hub 402. In certain embodiments, the catheter hub 404 is repositionable between an inserted position 424 as illustrated in FIG. 5A and an extended position 426 as illustrated in FIG. 5B.

In one embodiment, the catheter hub 404 includes a stopping surface 425. When the catheter hub 404 is fully inserted into the catheter hub receiving space 504, the stopping surface 425 of the catheter hub 404 contacts the sheath hub 402 to arrest further insertion of the catheter hub 404 within the catheter hub receiving space 504. With the catheter hub 404 fully positioned within the catheter hub receiving space 504 to the point where the stopping surface 425 contacts the sheath hub 402, the catheter hub 404 may be considered to be positioned in the fully inserted position 424 with the internal catheter being fully extended beyond the distal tip of the sheath. As the catheter hub 404 is withdrawn from within the catheter hub receiving space 504 in the direction indicated by arrows 407, the catheter hub 404 may be considered to be positioned in a fully extended position 426.

A catheter 412 is coupled to the catheter hub 404 at a coupling end 506 (see FIG. 6B) of the catheter 412. An insertion end (distal tip) 414 of the catheter 412 includes an intrinsic curvature 416 with respect to a longitudinal axis 418 of the catheter 412. The insertion end 414 of the catheter 412 is disposed opposite the coupling end 506 of the catheter 412. The intrinsic curvature 416 of the insertion end 414 of the catheter 412 causes the insertion end 414 of the catheter 412 to bend when advanced beyond the distal tip of the sheath 420 for delivery over the middle turbinate and to the SPG & V2 according to methods of the invention.

Because the catheter 412 is coupled to the catheter hub 404 and the sheath 420 is coupled to the sheath hub 402, when the catheter hub 404 is positioned in the extended position 426 the catheter 412 is withdrawn into the sheath 420 as illustrated in FIG. 5B. With the catheter 412 withdrawn into the sheath 420, the structural rigidity of the sheath 420 straightens the intrinsic curvature 416 of the insertion end 414 of the catheter 412 allowing the physician or other medical professional to manipulate the insertion end 414 of the catheter 412 past the anterior ridge 302 of the middle sinus turbinate 114.

Once the insertion end 414 of the catheter 412 has passed the anterior ridge 302 of the middle sinus turbinate 114, the physician or other medical professional can advance the catheter hub 404 to the inserted position 424. With the catheter hub 404 repositioned in the inserted position 424 the intrinsic curvature 416 of the insertion end 414 of the catheter 412 is not positioned within the sheath 420 and is therefore not straightened by the sheath 420. The intrinsic curvature 416 of the insertion end 414 of the catheter 412 causes the insertion end 414 of the catheter 412 to bend. The bend in the catheter 412 allows the physician or other medical professional to direct the insertion end 414 of the catheter 412 into the patient's sphenopalatine/pterygopalatine recess 118 where the physician or other medical professional can deliver treatment bilaterally to the SPG and V2.

FIG. 6A depicts a top view of one embodiment of an apparatus 400 for facilitating intranasal treatment of migraine pain. In the embodiment depicted FIG. 6A, the catheter hub 404 is positioned in the extended position 426. In certain embodiments, the sheath 420 includes an introduction end 505 that is sloped to a vertex 508 such that the entire introduction end 505 of the sheath 420 forms a smooth slope without any edges to catch on the tissue of the patient's nasal cavity 104.

In one embodiment, the insertion end 414 of the catheter 412 is curved such that a tip 510 of the insertion end 414 of the catheter 412 is rounded. By including a rounded tip 510 on the insertion end 414 of the catheter 412, the physician or other medical professional is less likely to catch or snag the delicate tissue of the patient's nasal cavity 104 with the insertion end 414 of the catheter 412. As further described below, in certain embodiments, when the catheter hub 404 is positioned in the extended position 426, the vertex 508 at the introduction end 505 of the sheath 420 aligns with a beginning of the curve of the rounded tip 510 of the catheter 412 such that a transition 512 between the catheter 412 and the sheath 420 is continuous, smooth and substantially edge free. A smooth transition 512 between the catheter 412 and the sheath 420 helps to avoid catching tissue within the patient's nasal cavity 104.

In certain embodiments, the treatment receiving port 436 includes a coupling member 514 for coupling the apparatus 400 to the treatment delivery device. For example, in one embodiment, the coupling member 514 may be a plurality of threads disposed around the circumference of the treatment receiving port 436. The threads of the coupling member 514 engage threads on a syringe or other treatment delivery device to couple the treatment delivery device to the treatment receiving port 436.

FIG. 6B depicts a side cutaway view of the second embodiment of an apparatus 400 for use with methods of the invention. In the embodiment depicted FIG. 5B, the catheter hub 404 is positioned in the extended position 426. The embodiment depicted in FIG. 5B is taken along line A-A of FIG. 5A and more clearly illustrates one embodiment of the interior surface 502 of the sheath hub 402 and the catheter hub receiving space 504.

In certain embodiments, the apparatus 400 includes an arresting element 516 on either the catheter hub 404 or the sheath hub 402. In the embodiment illustrated in FIG. 5B, the arresting element 516 is a flange that is coupled to and extends perpendicularly from an outer surface 518 of the reduced diameter portion 422 of the catheter hub 404.

In one embodiment, the apparatus 400 also includes an engagement element 520 on either the catheter hub 404 or the sheath hub 402. In the embodiment illustrated in FIG. 6B, the engagement element 520 is a recess extending longitudinally along the interior surface 502 of the sheath hub 402. The flange of the arresting element 516 is positioned within and travels along the recess of the engagement element 520 when the catheter hub 404 is repositioned along the longitudinal axis 428 of the sheath hub 402. Cooperation between the arresting element 516 and the engagement element 520 allows the catheter hub 404 to be slideably received within the catheter hub receiving space 504 while limiting rotation of the catheter hub 404 with respect to the sheath hub 402. Accordingly, in certain embodiments, the flange of the arresting element 516 is continuously engaged within the recess of the engagement element 520 when the catheter hub 404 is repositioned within along the longitudinal axis 428 of the sheath hub 402. Engagement between the arresting element 516 and the engagement element 520 prevents rotation of the sheath hub 402 with respect to the catheter hub 404.

In certain embodiments, the apparatus 400 also includes a stopping element 522 coupled to either the catheter hub 404 or the sheath hub 402. The stopping element 522 is configured to engage the arresting element 516 to stop the catheter hub 404 from being removed from the catheter hub receiving space 504. In the embodiment illustrated in FIG. 6B, the stopping element 522 is a substantially rigid wall that engages the arresting element 516 to stop the catheter hub 404 from being removed from the catheter hub receiving space 504. In certain embodiments, the stopping element 522 also facilitates alignment of the vertex 508 at the introduction end 505 of the sheath 420 with the beginning of the curve of the rounded tip 510 of the catheter 412 such that the transition 512 between the catheter 412 and the sheath 420 is continuous, smooth and substantially edge free when the catheter hub 404 is positioned in the extended position 426.

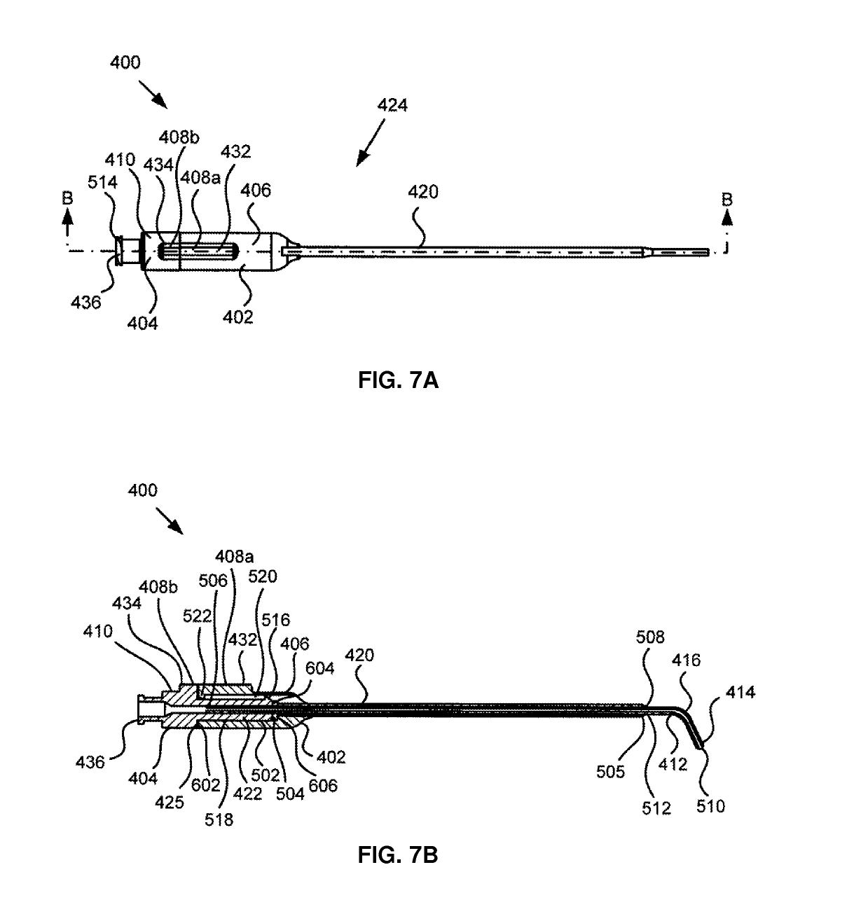

FIG. 7A depicts a top view of one embodiment of an apparatus 400 for facilitating intranasal treatment of a patient's sphenopalatine/pterygopalatine recess 118. In the embodiment illustrated in FIG. 7A, the catheter hub 404 is positioned in the inserted position 424.

FIG. 7B is a side cutaway view of one embodiment of an apparatus for facilitating intranasal treatment of a patient's sphenopalatine/pterygopalatine recess 118. In the embodiment depicted FIG. 7B, the catheter hub 404 is positioned in the inserted position 424. The embodiment depicted in FIG. 7B is taken along line B-B of FIG. 7A.

In one embodiment, when the catheter hub 404 is fully inserted into the catheter hub receiving space 504, the stopping surface 425 of the catheter hub 404 contacts an end 602 of the sheath hub 402 to arrest further insertion of the catheter hub 404 within the catheter hub receiving space 504. In other embodiments, an end 604 of the reduced diameter portion 422 of the catheter hub 404 contacts an interior wall 606 within the catheter hub receiving space 504 to arrest further insertion of the catheter hub 404 within the catheter hub receiving space 504. With the catheter hub 404 fully positioned within the catheter hub receiving space 504 to the point where the stopping surface 425 contacts the sheath hub 402, the catheter hub 404 may be considered to be positioned in the fully inserted position 424.

It is noted that FIGS. 5A-7B show a device 424 and in cutaway views or cross-section illustrated mechanism 400 by which drug delivery is accomplished. The depicted cutaway views illustrate mechanical components also found in device 101 and drug delivery therefrom may operate via the same principles. Additional useful background may be found in U.S. Pub. 2012/0157968, incorporated by reference.

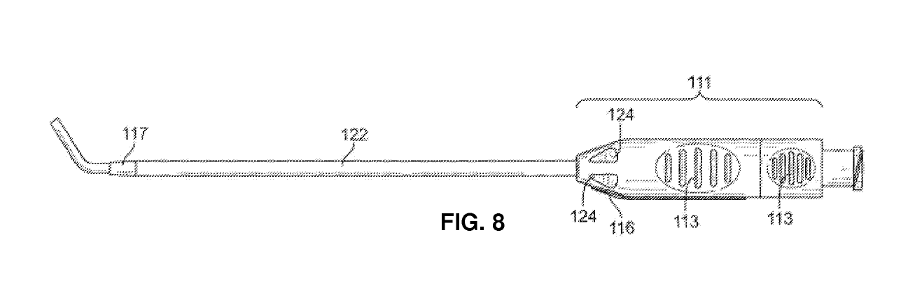

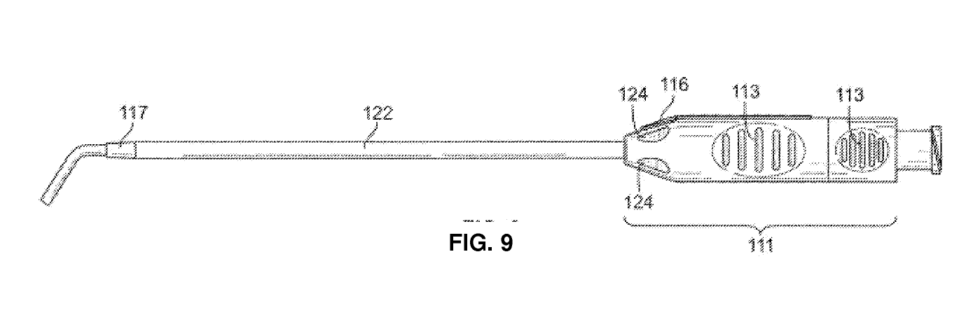

FIG. 8 and FIG. 9, each side views of a drug delivery device 101, demonstrate ergonomic hub/handle 111 with further gripping features 124 and the relationship between directional arrow 116 and deflecting tip/medication delivery port 123—namely, arrow 116 shows orientation of deflecting tip/medication delivery port 123 which angles shown by arrow 138 allows deflecting tip 123 to bend in the direction of a patient's SPG to deliver, for example, CGRP and the like actives and medications.

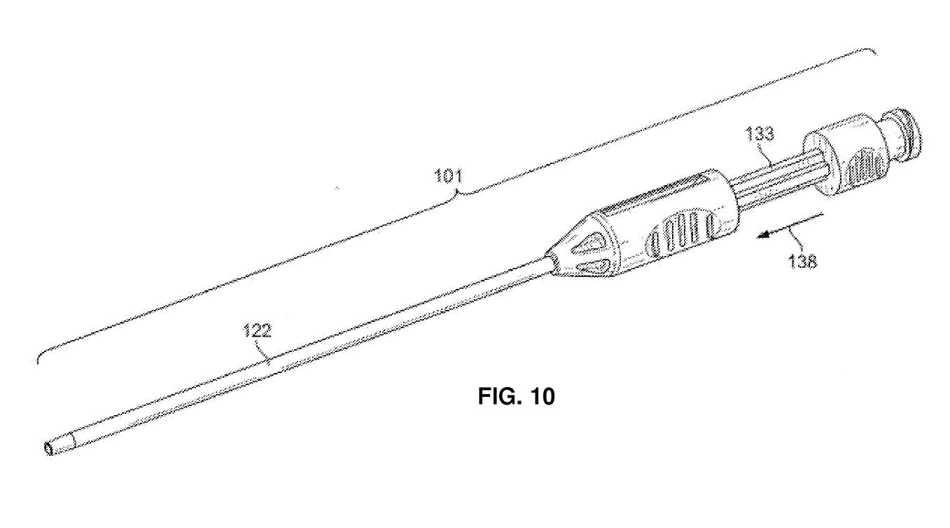

Referring still to FIG. 1 and FIG. 10, the Sphenocath® brand of device 101 is emplaced while patient is supine, and extends in a first position, past the middle turbinate 100 to access the pterygopalatine/sphenopalatine fossa 102, which is adjacent to the SPG 99. As shown in FIG. 10, direction 138 shows how medication travels down the path as the catheter moves from a first position to a second position, where stiffening member 133 is extended.

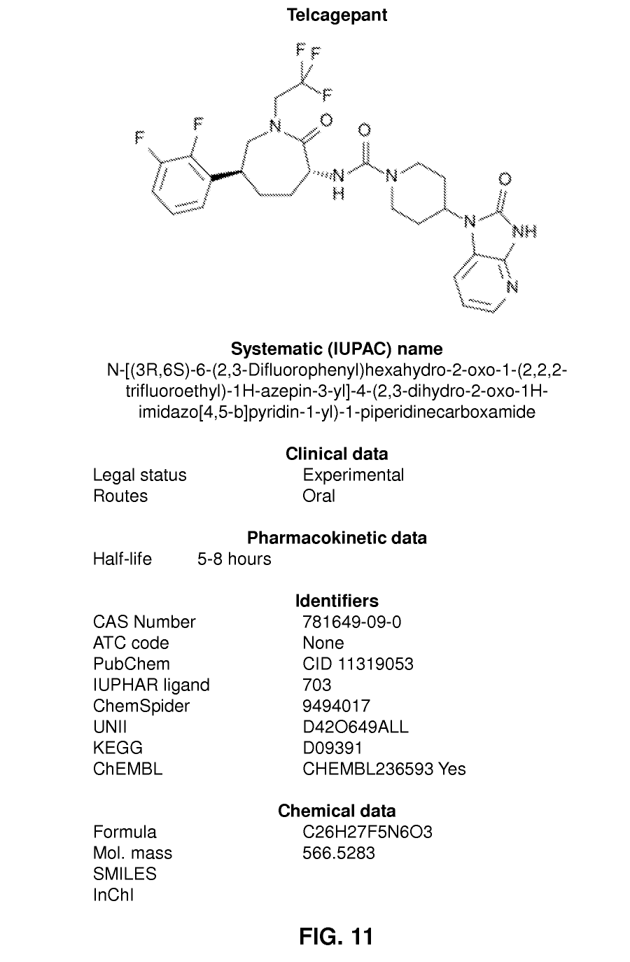

FIG. 11 shows a CGRP antagonist that may be used as a component of the medication in methods of the invention. Namely, diminished amounts of this moiety, or others, when directly delivered to the SPG circuit, function as CGRP agonist.

Methods of the invention provide delivery with localization to the SPG and V2. This is different from the temporary relief of a nerve block, because it can impact the multiple pathways connected to the SPG, via mechanisms such as SPCG. By addressing placement and delivery of medication to the SPG, as opposed to quasi-candon spraying of short-term agents, a new paradigm in pain relief has been entered. This standard of care shall likewise presently and directly be adopted and provides relief to patients, some of whom have suffered for years without relief.

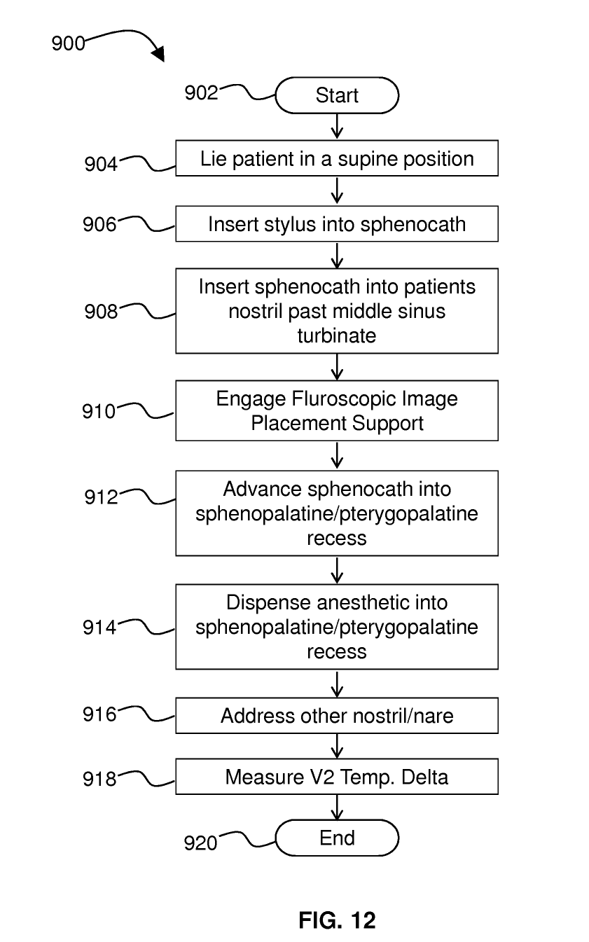

FIG. 12 diagrams a method 900 of determining a cutaneous temperature increase over the V2 dermatomal distribution of the face as a metric of successful delivery of anesthesia to the SPG and V2. Method 900 starts 902 by having the patient lie 904 in the supine position. A stylus is inserted 906 into the delivery device and the delivery device is inserted 908 into the patient's nostril past the middle sinus turbinate. The physician may engage 910 fluoroscopic image placement support and advance 912 the delivery device into the SPG recess. An anesthesia is dispensed 914 into the SPG recess, after or during which the physician also addresses 916 the other recess. The physician then measures 918 a change in temperature of the V2 dermatomal distribution of the face to determine that the procedure should end 920 for being successful as indicated by a delta temperature of about 3 to 5 degrees.

In a preferred embodiment, the invention provides a method for treating pain by bilateral medication of the SPG and V2.

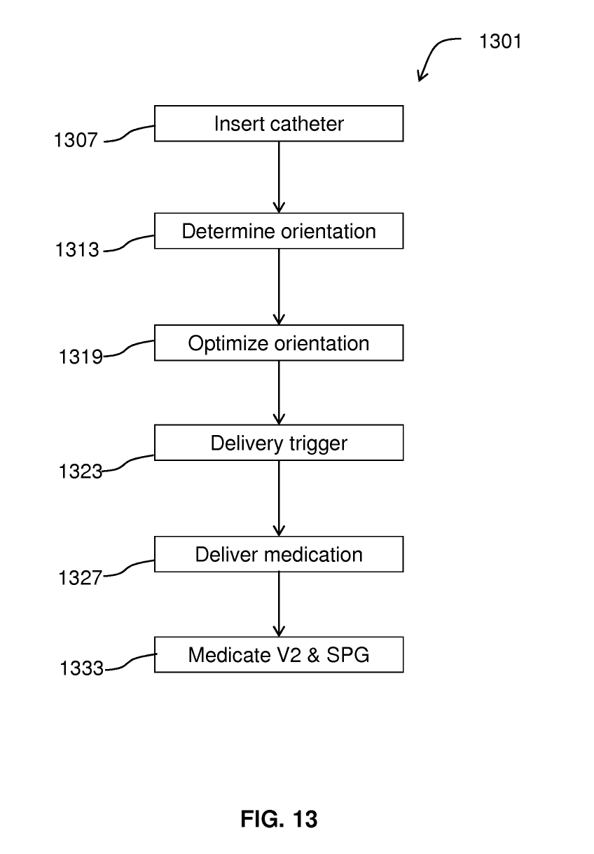

FIG. 13 describes a method 1301 of treating a patient for pain. The method includes inserting 1307 at least one catheter of a drug delivery device into a nostril of a patient lying supine.

In a preferred embodiment, the drug delivery device includes the catheter defining an extended body with the distal end including an exit port. The device includes a handle at the proximal portion of the catheter, at least one lumen in the catheter (e.g., wherein movement of an inner member delivers the medication through the lumen and out of the exit port at the distal portion of the catheter), and a reservoir within the device for holding the medication

Optionally, the operator/physician may determine 1313 the orientation of the curved portion of the catheter member (e.g., by touching a “reminder” marker on the handle) and optimize 1319 the orientation. The physician can optimize the orientation by twisting the handle to direct the exit port over the middle turbinate and into the area of the SPG and V2.

The method 1301 further includes operating 1323 a delivery mechanism on a proximal portion of the drug delivery device to cause a medication to be pushed in a distal direction through the catheter. This leads to delivering 1327 the medication out of an exit port on a distal end of the catheter and into a target site in the patient, thereby medicating 1333 the V2 and SPG by causing the medication to make contact with the V2 and the SPG.

Numerous definitional aspects of the “sphenopalatine circuit blockade” or SPCG defined as bilateral SPG/PPG/V2 have yet to become generally accepted and understood. To the extent possible, harmonizing terms and descriptive terminology is offered for consideration to differentiate legacy terms from the state of the art.

Both acute and chronic headaches have serious impacts upon the ability of people to function. The numerous classification systems, calling various atypical headaches by labels from cluster headaches to trigeminal neuralgia, tension, or anything else modulated by the sphenopalatine ganglion (SPG) have one universal—the need for immediate and accurate placement of medication to address them.

As detailed herein, “neuromodulation” means a physiological response reflecting long-term changes persisting beyond the half-life of local anesthetics. Likewise, the SPG is defined as that ganglion, or set of nerves grouped together, which sends messages to other nerves, which is located in the back of the nasal cavity and can only be accessed (non-surgically) by dispensing medication over the top of the middle sinus turbinate. “SPG Circuit Blockade” (SPCB) means delivery to all elements of the SPG circuit of medication which includes a pain treatment compound such as CGRP agonists, like that shown in FIG. 10. We have created a term called “Sphenopalatine Circuit Blockade” or (SPCB), and we define as bilateral blockage treatment of SPG/PPG/V2.

As a result of this exquisite contact of injectate with V2 and the SPG, the SphenoCath® not only addresses all of the major pain initiators in the migraine and TAC related disorders, it provides an exquisite treatment modality for trigeminal neuralgia, and other atypical facial and head pain syndromes. Additionally, the SphenoCath® can now be used to treat dental pain that has otherwise previously been hard to achieve.

All of the aforementioned treatment modalities are enhanced when the SphenoCath® inner purple curved catheter is directed towards the side wall of the nasal-sinus passageway over the middle turbinate and under the superior turbinate. As such, we include and claim herein additional methods, which describe a more lateral injectate disbursement. Because the directional apparatus on the SphenoCath® hub and the inner curved catheter, are in oppositional planar relationships, we are now instructing practitioners to rotate the directional apparatus marker on the hub towards the midline of the nose approximately 10° on each nostril. This facilitates the lateral disbursement of injectate along the side wall of the nasal-sinus passageway above the middle turbinate and below the superior. This understanding of the anatomy is known by no one but dolor technologies. And it is essential to define this procedural adjustment in our intellectual property.

Likewise, we call attention to the method of determining a 3-5° cutaneous temperature increase over the V2 dermatomal distribution of the face as a metric of successful SPCB procedural application. We are using micro crystalline skin temperature stickers placed over the cheek in the V2 distribution to document the initial skin temperature (usually 93-94 degrees), increasing in temperature to 97-99°, post-procedural. A wonderful article been crafted by world class migraine neurologists is forthcoming describing the science behind this. The reader's digest version of how this occurs relates to a parasympathetic overdrive (vasodilation), superimposed by somatosensory micro circulatory enhanced perfusion. The take home message is to dilate the blood flow, and bring core temperature to the surface. No other procedural application can accomplish this besides the SphenoCath®.

Calcitonin gene-related peptide (CGRP) is a well-studied neuropeptide found at the very centers of the migraine processes, CGRP receptor antagonists disrupt the interaction of CGRP with its receptor and are being developed primarily for the acute treatment of migraine. Free CGRP and CGRP receptors can also be targeted using monoclonal antibodies which are being developed. Any suitable CGRP receptor antagonist may be used with devices, systems, and methods of the invention. For example, the CGRP receptor antagonist may include one of the CGRP receptor antagonists known as the ‘gepants’ that have been shown to have efficacy for the acute treatment of migraine.

One of the gepants—olcegepant (BIBN4096BS)—was discontinued because of difficulties in developing an oral formulation but may used with a delivery device of the invention (Olesen et al., 2004, Calcitonin gene related peptide receptor antagonist BIBN 4096 BS for the acute treatment of migraine. NEJM 350:1104-1110). Telcagepant (MK-0974) is a gepant that was discontinued because of concerns of liver toxicity after frequent use (Silberstein, 2013, Emerging target-based paradigms to prevent and treat migraine. Clin Pharmacol Ther 93:78-85; Hoffmann and Goadsby, 2012, New Agents for Acute Treatment of Migraine: CGRP Receptor Antagonists, iNOS Inhibitors. Curr Treat Options Neurol 14:50-59). MK-3207, a gepant molecule that was significantly more potent than telcagepant, was also discontinued because of concerns of liver toxicity (Salvatore et al., 2010, Pharmacological properties of MK-3207, a potent and orally active calcitonin gene-related peptide receptor antagonist. J Pharmacol Exp Ther 333:152-160; Pettypiece S., 2009, Merck Halts Testing of Migraine Drug on Liver Safety, Update2). The gepant BI44370A had efficacy demonstrated in a Phase 2 clinical study and may be delivered using devices and systems of the invention. (Diener et al., 2011, BI 44370 TA, an oral CGRP antagonist for the treatment of acute migraine attacks: Results from a phase II study. Cephalalgia 31:573-584). In addition to demonstrating proof of efficacy, CGRP receptor antagonist clinical trials demonstrated the tolerability of the class with acute dosing and that, as opposed to triptans, their use is not associated with vasoconstriction.

A fifth ‘gepant’ just joined the club by its own merits. In Cephalalgia 34(2):114-25 (2014), Marcus and colleagues report the result of a large Phase 2b study testing BMS-927711 for the acute treatment of migraine and that compound may be delivered using devices and systems of the invention. The primary efficacy endpoint was the proportion of pain-free subjects at 2 hours post dose. In addition to testing the primary endpoint for statistical significance compared with placebo, the authors attempted to define a measure of clinical relevance, or clinical response of at least 15% greater than the response of placebo. (Bigal, M., “BMS-927711 for the acute treatment of migraine” Cephalalgia 2014, Vol. 34 (2) pp. 90-92.)

The literature suggests that CGRP receptor antagonists are effective at multiple doses, meaning that all CGRP receptor antagonists tested to date have demonstrated efficacy in Phase 2 and Phase 3 studies.

Efficacy was established by the authors, although it was found to be numerically inferior to sumatriptan for this endpoint for all doses (although the study was not powered for direct comparisons). The drug also passed the bar of the secondary endpoints where most (and sometimes all) doses were superior to placebo, and most effective doses were also numerically superior to sumatriptan. Accordingly, certain doses of BMS-927711 seem to deliver levels of efficacy that are similar to those delivered by the highest dose of an effective triptan. Overall, the efficacy of the drug increased up to the dose of 75 mg when a plateau effect seemed to have been reached for doses between 75 mg and 300 mg. The highest tested dose (600 mg) demonstrated no additional benefit over the doses of 75 mg and 150 mg.

The most effective doses of the drug also significantly improved photophobia and phonophobia, which are known to be SPG-medicated results of many pain types.

The tolerability of BMS-927711 was reported as being placebo-like, which once more the authors conclude, reinforces the tolerability of the class for acute dosing. As with the other CGRP receptor antagonists, a pattern of side effects could not be identified and this is different from what is seen for other classes such as ergot derivatives and triptans (where certain adverse events, such as chest tenderness and muscle tightness seem to be class-specific). No serious adverse event was reported in the trial.

In sum, the efficacy of a single dose of BMS-927711 has been demonstrated. In this regard, one would expect Phase 3 to focus on defining consistency of efficacy when treating multiple attacks. Tolerability also seems to be consistent with expectations for the class, and one would expect Phase 3 to reinforce the finding. Accordingly, Phase 3 would be mainly about defining the safety of the drug, especially its effect on the liver in situations of frequent dosing and in patients using medications that are metabolized by the CYP3A4. A drug that has sumatriptan-like efficacy, that may be better tolerated, and without vasoconstrictive properties, would be an incredible addition to the migraine treatment arsenal, clearly addressing current unmet needs. In particular, patients with existing vascular disease or vascular risk factors currently have only very limited choices of acute migraine medications.

Any other suitable CGRP receptor antagonist may be used in systems and methods of the invention. For example, N-[1-(2,3-Difluorobenzyl)-5-oxo-4-(2,22-trifluoroethyl)-1,4-diazepan-6-yl]-4-(2-oxo-2,3-dihydro-1H-imidazo[4,5-b]pyridin-1-yl)piperidine-1-carboxamide may be used. Other CGRP receptor antagonists suitable for use with the invention include those discussed in U.S. Pat. No. 7,772,224 to Paone; U.S. Pub. 2005/0215576 to Degnan; U.S. Pub. 2007/0148093 to Conway; U.S. Pat. No. 7,220,862 to Chaturvedula; and U.S. Pub. 2004/0063735, the contents of each of which are incorporated by reference for all purposes.

While methods, devices, compositions, and the like, have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims. It is understood that the term, present disclosure, in the context of a description of a component, characteristic, or step, of one particular embodiment of the disclosure, does not imply or mean that all embodiments of the disclosure comprise that particular component, characteristic, or step.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these disclosure(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC §132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular implementation, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps. Such terms should be interpreted in their most expansive forms so as to afford the applicant the broadest coverage legally permissible.

What is claimed is:

1. A method of treating a patient for pain, the method comprising:
inserting at least one catheter of a drug delivery device into a nostril of a patient lying supine;
operating a delivery mechanism on a proximal portion of the drug delivery device to cause a medication to be pushed in a distal direction through the catheter;
delivering the medication out of an exit port on a distal end of the catheter and into a target site in the patient; and
medicating a maxillary branch of a trigeminal nerve (V2) and a sphenopalatine ganglion (SPG) by causing the medication to make contact with the V2 and the SPG.

2. The method of claim 1, wherein the drug delivery device includes: the catheter defining an extended body with the distal end, wherein the distal end includes a curved portion and the exit port;
a handle at the proximal portion of the catheter;
at least a first lumen in the catheter, wherein movement of an inner member delivers the medication through the first lumen and out of the exit port at the distal portion of the catheter; and
a reservoir within the device for holding the medication.

3. The method of claim 2, wherein the proximal end of the drug delivery device further comprises a marking on the handle that indicates an orientation of the curved portion of the catheter member.

4. The method of claim 3, further comprising:
determining the orientation of the curved portion of the catheter member; and
optimizing the orientation.

5. The method of claim 4, wherein determining the orientation includes touching the marking on the handle and optimizing the orientation includes manipulating the handle.

6. The method of claim 5, further comprising instructing the patient to remain supine for at least a minute to allow the medication to migrate into a pterygopalatine fossa.

7. The method of claim 6, wherein the inserting and operating steps are performed for each of two nostrils of the patient.

8. The method of claim 7, further comprising causing the medication to make bilateral contact with both V2 and both SPG of the patient.

9. The method of claim 8, wherein the medication comprises a neuromodulator.

10. The method of claim 8, wherein the medication comprises an anesthetic.

11. The method of claim 8, wherein the medication comprises a calcitonin gene-related peptide (CGRP) receptor antagonist

12. The method of claim 8, wherein the drug delivery device further comprises at least a radiopaque marking band located at the distal portion of the catheter.

13. The method of claim 8, wherein the handle further comprises a gripping surface with textured portions for grasping the drug delivery device.

14. The method of claim 8, wherein the medication comprises means for delivery of agents effective for repolarization of cranial sensory and parasympathetic pathways via trigeminal primary afferent neurons.

15. The method of claim 8, wherein the drug delivery device further comprises a plunger member operable to apply pressure to the medication in the drug delivery device.

15. The method of claim 8, wherein the marking on the handle comprises a three-dimensional indicia configured to be detected via touch by a hand of an operator to indicate the orientation of the curved distal end of the catheter.

16. The method of claim 15, wherein the three-dimensional indicia is shaped to represent the curved distal end of the catheter.

17. The method of claim 8, further comprising viewing instructions describing the method on information materials provided with the drug delivery device.
